SEQUENCE LISTING (2 sequences)

SEQ ID NO: 1 - protein, 1069 aa, Homo sapiens
MLRMRTAGWARGWCLGCCLLLPLSLSLAAAKQLLRYRLAEEGPADVRIGNVASDLGIVTGSGEVTFSLESGSEYLKIDNLTGELSTSERRIDREKLPQCQMIFDENECFLDFEVSVIGPSQSWVDLFEGQVIVLDINDNTPTFPSPVLTLTVEENRPVGTLYLLPTATDRDFGRNGIERYELLQEPGGGGSGGESRRAGAADSAPYPGGGGNGASGGGSGGSKRRLDASEGGGGTNPGGRSSVFELQVADTPDGEKQPQLIVKGALDREQRDSYELTLRVRDGGDPPRSSQAILRVLITDVNDNSPRFEKSVYEADLAENSAPGTPILQLRAADLDVGVNGQIEYVFGAATESVRRLLRLDETSGWLSVLHRIDREEVNQLRFTVMARDRGQPPKTDKATVVLNIKDENDNVPSIEIRKIGRIPLKDGVANVAEDVLVDTPIALVQVSDRDQGENGVVTCTVVGDVPFQLKPASDTEGDQNKKKYFLHTSTPLDYEATREFNVVIVAVDSGSPSLSSNNSLIVKVGDTNDNPPMFGQSVVEVYFPENNIPGERVATVLATDADSGKNAEIAYSLDSSVMGIFAIDPDSGDILVNTVLDREQTDRYEFKVNAKDKGIPVLQGSTTVIVQVADKNDNDPKFMQDVFTFYVKENLQPNSPVGMVTVMDADKGRNAEMSLYIEENNNIFSIENDTGTIYSTMSFDREHQTTYTFRVKAVDGGDPPRSATATVSLFVMDENDNAPTVTLPKNISYTLLPPSSNVRTVVATVLATDSDDGINADLNYSIVGGNPFKLFEIDPTSGVVSLVGKLTQKHYGLHRLVVQVNDSGQPSQSTTTLVHVFVNESVSNATAIDSQIARSLHIPLTQDIAGDPSYEISKQRLSIVIGVVAGIMTVILIILIVVMARYCRSKNKNGYEAGKKDHEDFFTPQQHDKSKKPKKDKKNKKSKQPLYSSIVTVEASKPNGQRYDSVNEKLSDSPSMGRYRSVNGGPGSPDLARHYKSSSPLPTVQLHPQSPTAGKKHQAVQDLPPANTFVGAGDNISIGSDHCSEYSCQTNNKYSKQMRLHPYITVFG

SEQ ID NO: 2 - protein, 13 aa, Homo sapiens
LDETSGWLSVLHR

(19) United States
(12) Patent Application Publication
(10) Pub. No.: US 2023/0314425 A1
(43) Pub. Date: Oct. 5, 2023

(54) METHODS AND MATERIALS FOR IDENTIFYING AND TREATING MEMBRANOUS NEPHROPATHY

(71) Applicant: Mayo Foundation for Medical Education and Research, Rochester, MN (US)

(72) Inventors: Sanjeev Sethi, Rochester, MN (US); Fernando C. Fervenza, Rochester, MN (US); Benjamin J. Madden, Stewartville, MN (US); M. Cristine Charlesworth, Rochester, MN (US)

(21) Appl. No.: 18/021,641

(22) PCT Filed: Sep. 1, 2021
(86) PCT No.: PCT/US21/48595

§ 371 Date: Feb. 16, 2023

Related U.S. Application Data
(60) Provisional application No. 63/073,339, filed on Sep. 1, 2020.

(51) Int. Cl. G01N 33/564; A61K 45/06

(52) U.S. Cl. CPC G01N 33/564; A61K 45/06; G01N 2800/347

(57) ABSTRACT

This document relates to methods and materials involved in identifying and/or treating mammals having membranous nephropathy (e.g., membranous nephropathy with an elevated level of a PCDH7 polypeptide in the glomerular basement membrane (GBM)). For example, methods and materials for administering one or more immunosuppressive agents (e.g., corticosteroids, cyclosporine, or a B-cell reduction or depletion agent such as Rituximab) to treat a mammal (e.g., a human) having membranous nephropathy are provided.

### CROSS-REFERENCE TO RELATED APPLICATIONS

This application claims the benefit of U.S. Patent Application Ser. No. 63/073,339, filed on Sep. 1, 2020. The disclosure of the prior application is considered part of (and is incorporated by reference in) the disclosure of this application.

### SEQUENCE LISTING

This document contains a Sequence Listing that has been submitted electronically as an ASCII text file named 07039-1992WO1 ST25.txt. The ASCII text file, created on Aug. 13, 2021, is 10 kilobytes in size. The material in the ASCII text file is hereby incorporated by reference in its entirety.

### BACKGROUND

### 1. Technical Field

This document relates to methods and materials involved in identifying and/or treating mammals having membranous nephropathy (e.g., membranous nephropathy with an elevated level of a protocadherin-7 (PCDH7) polypeptide in the glomerular basement membrane (GBM)). For example, this document provides methods and materials for administering one or more immunosuppressive agents (e.g., corticosteroids, cyclosporine, or a B-cell reduction or depletion agent such as Rituximab) to treat a mammal (e.g., a human) having membranous nephropathy.

### 2. Background Information

Membranous nephropathy (MN) is the most common cause of nephrotic syndrome in Caucasian adults. It is caused by autoantibodies against target antigens in the glomerular basement membrane (GBM), and is characterized by antigen-antibody complexes that form deposits along the GBM. MN can be classified based on the target antigens phospholipase A2 receptor 1 (PLA2R), thrombospondin type-1 domain-containing protein 7A (THSD7A), neural epidermal growth factor-like 1 (NELL-1), and Semaphorin 3B (Sema3B). For example, MN can be classified as PLA2R-positive (70%), THSD7A-positive (1-5%), NELL-1 (5%), Sema3b (1%) or PLA2R/THSD7A/NELL-1/Sema3B-quadruple negative MN. In the PLA2R/THSD7A/NELL-1/Sema3B-quadruple negative MN, the target antigen(s) remain elusive.

### SUMMARY

This document provides methods and materials involved in identifying and/or treating mammals (e.g., humans) having membranous nephropathy (e.g., membranous nephropathy with an elevated level of a PCDH7 polypeptide in the GBM). For example, this document provides methods and materials for identifying a mammal (e.g., a human) having membranous nephropathy having an elevated level of a PCDH7 polypeptide in the GBM that can serve as a target antigen in membranous nephropathy. This document also provides methods and materials for identifying a mammal (e.g., a human) having membranous nephropathy that includes the presence of autoantibodies having binding specificity for a PCDH7 polypeptide. As described herein, mammals (e.g., humans) having membranous nephropathy can be identified as having an elevated level of a PCDH7 polypeptide in the GBM. In such cases, the mammal can be classified as having membranous nephropathy that includes an elevated level of a PCDH7 polypeptide in the GBM. As also described herein, mammals (e.g., humans) having membranous nephropathy can be identified as having autoantibodies having binding specificity for a PCDH7 polypeptide. In such cases, the mammal can be classified as having membranous nephropathy that includes the presence of autoantibodies having binding specificity for a PCDH7 polypeptide. Identifying mammals (e.g., humans) as having membranous nephropathy that includes an elevated level of a PCDH7 polypeptide in the GBM and/or that includes the presence of autoantibodies having binding specificity for a PCDH7 polypeptide can allow clinicians and patients to proceed with appropriate membranous nephropathy treatment options.

This document also provides methods and materials for treating membranous nephropathy. For example, a mammal (e.g., a human) having membranous nephropathy that was identified as having an elevated level of a PCDH7 polypeptide in the GBM, as having autoantibodies having binding specificity for a PCDH7 polypeptide, or as having both an elevated level of a PCDH7 polypeptide in the GBM and autoantibodies having binding specificity for a PCDH7 polypeptide can be administered one or more immunosuppressive agents (e.g., corticosteroids, cyclosporine, or a B-cell reduction or depletion agent such as Rituximab) to reduce inflammation and/or B-cell autoantibody production. As described herein, mammals (e.g., humans) having membranous nephropathy and identified as having an elevated level of a PCDH7 polypeptide in the GBM and/or as having autoantibodies having binding specificity for a PCDH7 polypeptide have a form of membranous nephropathy that is caused by the presence of antigen-autoantibody complexes where the antigen is a PCDH7 polypeptide. In such cases, the mammal (e.g., human) can be effectively treated using one or more immunosuppressive agents (e.g., corticosteroids, cyclosporine, or a B-cell reduction or depletion agent such as Rituximab) to reduce inflammation and/or B-cell autoantibody production. Having the ability to administer one or more immunosuppressive agents to mammals (e.g., humans) (a) having membranous nephropathy and (b) identified as having an elevated level of a PCDH7 polypeptide in the GBM and/or as having autoantibodies having binding specificity for a PCDH7 polypeptide can allow clinicians and patients to treat membranous nephropathy effectively.

As also described herein, most, if not all, membranous nephropathy cases in humans are caused by autoantibodies having specificity to a polypeptide that accumulates in the GBM. Those polypeptides include PCDH7, Semaphorin 3B, neural epidermal growth factor (EGF)-like 1 (NELL-1), exostosin 1 (EXT1), exostosin 2 (EXT2), PLA2R, and THSD7A. In general, the use of immunosuppressive agents such as B-cell reduction or depletion agents (e.g., Rituximab) in cases such as membranous nephropathy currently requires an identification of autoantibodies (e.g., anti-PLA2R autoantibodies or anti-THSD7A autoantibodies) before a powerful B-cell reduction or depletion agent such as Rituximab can be administered to a human to treat membranous nephropathy. Based, at least in part, on the results presented herein, however, such an identification is no longer needed prior to using an immunosuppressive agent to treat membranous nephropathy. For example, a mammal (e.g., a human) having membranous nephropathy (e.g., membranous nephropathy with an elevated level of a PCDH7, a Semaphorin 3B, a NELL-1, an EXT1, an EXT2, a PLA2R, and/or a THSD7A polypeptide) can be administered one or more immunosuppressive agents (e.g., corticosteroids, cyclosporine, or a B-cell reduction or depletion agent such as Rituximab) to treat membranous nephropathy without having been tested for an elevated level of any polypeptide in the GBM and without having been tested for the presence of any autoantibody. In some cases, a mammal (e.g., a human) having membranous nephropathy can be administered one or more immunosuppressive agents (e.g., corticosteroids, cyclosporine, or a B-cell reduction or depletion agent such as Rituximab) to treat membranous nephropathy without having been tested for an elevated level of (a) a PCDH7 polypeptide, (b) a Semaphorin 3B polypeptide, (c) a NELL-1 polypeptide, (d) an EXT1 polypeptide, (e) an EXT2 polypeptide, (f) a PLA2R polypeptide, and (g) a THSD7A polypeptide and without having been tested for the presence of (a) an autoantibody having specificity for a PCDH7 polypeptide (b) an autoantibody having specificity for a Semaphorin 3B polypeptide, (c) an autoantibody having specificity for a NELL-1 polypeptide, (d) an autoantibody having specificity for an EXT1 polypeptide, (e) an autoantibody having specificity for an EXT2 polypeptide, (f) an autoantibody having specificity for a PLA2R polypeptide, and (g) an autoantibody having specificity for a THSD7A polypeptide. Having the ability to treat membranous nephropathy without prior testing for elevated levels of particular polypeptides in the GBM and without prior testing for the presence of particular autoantibodies can allow clinicians and patients to treat membranous nephropathy safely without the added testing delay or expense.

In some cases, identification of the target antigen and autoantibodies can be involved in the diagnosis and/or management of a mammal (e.g., a human) with membranous nephropathy. For example, a mammal (e.g., a human) having membranous nephropathy (e.g., membranous nephropathy with GBM accumulation of a PCDH7, Semaphorin 3B, NELL-1, EXT1, EXT2, PLA2R, and/or THSD7A polypeptide and the presence of autoantibodies to one or more target antigens) can be administered one or more immunosuppressive agents (e.g., corticosteroids, cyclosporine, or a B-cell reduction or depletion agent such as Rituximab) to treat membranous nephropathy. In some cases, the response to the immunosuppressive treatment can be monitored for a decrease or complete elimination of the autoantibodies to one or more of a PLA2R, THSD7A, EXT1, EXT2, NELL-1, Semaphorin 3B, or PCDH7 polypeptide. In some cases, the response to treatment can be monitored by examining a kidney biopsy for a decrease or elimination of one or more target antigens (e.g., a PLA2R, THSD7A, EXT1, EXT2, NELL-1, Semaphorin 3B, or PCDH7 polypeptide). In some cases, a mammal (e.g., a human) having membranous nephropathy can be administered one or more immunosuppressive agents (e.g., corticosteroids, cyclosporine, or a B-cell reduction or depletion agent such as Rituximab) to treat membranous nephropathy based on the presence of an autoantibody to one or more of a PLA2R, THSD7A, EXT1, EXT2, NELL-1, Semaphorin 3B, or PCDH7 polypeptide in the absence of evaluating a kidney biopsy for an elevated level of a PLA2R, THSD7A, EXT1, EXT2, NELL-1, Semaphorin 3B, or PCDH7 polypeptide. Although kidney biopsies showing an accumulation of PLA2R, THSD7A, EXT1, EXT2, NELL-1, Semaphorin 3B, and/or PCDH7 polypeptides in GBM may be considered a gold standard for diagnosis of membranous nephropathy, the presence of autoantibodies to a PLA2R, THSD7A, EXT1, EXT2, NELL-1, Semaphorin 3B, or PCDH7 polypeptide can be used to identify specific types of membranous nephropathy (e.g., membranous nephropathy associated with accumulation of PLA2R, THSD7A, EXT1, EXT2, NELL-1, Semaphorin 3B, or PCDH7 polypeptides) without the need for a kidney biopsy.

In general, one aspect of this document features methods for identifying a mammal as having membranous nephropathy (e.g., active membranous nephropathy) including an elevated level of a polypeptide within kidney tissue of the mammal, where the polypeptide is a PCDH7 polypeptide. The methods can include, or consist essentially of, (a) determining the presence or absence of autoantibodies within the mammal, where the autoantibodies are specific for the polypeptide, (b) classifying the mammal as having the membranous nephropathy if the autoantibodies are present within the mammal, and (c) classifying the mammal as not having the membranous nephropathy if the autoantibodies are absent within the mammal. The mammal can be a human. The membranous nephropathy can lack an elevated level of a NELL-1 polypeptide within the kidney tissue, can lack an elevated level of a Semaphorin 3B polypeptide within the kidney tissue, can lack an elevated level of an EXT1 polypeptide within the kidney tissue, and/or can lack an elevated level of an EXT2 polypeptide within the kidney tissue. The membranous nephropathy can lack an elevated level of a PLA2R polypeptide within the kidney tissue. The membranous nephropathy can lack an elevated level of a THSD7A polypeptide within the kidney tissue. The method can include detecting the presence of the autoantibodies and classifying the mammal as having the membranous nephropathy. The method can include detecting the absence of the autoantibodies and classifying the mammal as not having the membranous nephropathy.

In another aspect, this document features methods for identifying a mammal as having kidney tissue including an elevated level of a polypeptide, where the polypeptide is a PCDH7 polypeptide. The methods can include, or consist essentially of, (a) determining the presence or absence of the kidney tissue within a sample obtained from the mammal, (b) classifying the mammal as having the kidney tissue if the presence is determined, and (c) classifying the mammal as not having the kidney tissue if the absence is determined. The mammal can be a human. The kidney tissue can lack an elevated level of a NELL-1 polypeptide, can lack an elevated level of an EXT1 polypeptide, can lack an elevated level of an EXT2 polypeptide, and/or can lack an elevated level of a Semaphorin 3B polypeptide. The kidney tissue can lack an elevated level of a PLA2R polypeptide. The kidney tissue can lack an elevated level of a THSD7A polypeptide. The method can include detecting the presence and classifying the mammal as having the kidney tissue. The method can include detecting the absence and classifying the mammal as not having the kidney tissue.

In another aspect, this document features methods for identifying a mammal having membranous nephropathy as having autoantibodies specific for a polypeptide, where the polypeptide is a PCDH7 polypeptide. The methods can include, or consist essentially of, (a) determining the presence or absence of the autoantibodies within the mammal, (b) classifying the mammal as having the autoantibodies if the autoantibodies are present within the mammal, and (c) classifying the mammal as not having the autoantibodies if the autoantibodies are absent within the mammal. The mammal can be a human. The kidney tissue of the mammal can lack an elevated level of a NELL-1 polypeptide, can lack an elevated level of an EXT1 polypeptide, can lack an elevated level of an EXT2 polypeptide, and/or can lack an elevated level of a Semaphorin 3B polypeptide. The kidney tissue of the mammal can lack an elevated level of a PLA2R polypeptide. The kidney tissue of the mammal can lack an elevated level of a THSD7A polypeptide. The method can include detecting the presence and classifying the mammal as having the autoantibodies. The method can include detecting the absence and classifying the mammal as not having the autoantibodies.

In another aspect, this document features methods for treating a mammal having membranous nephropathy. The methods can include, or consist essentially of, (a) identifying a mammal as having (i) autoantibodies specific for a polypeptide or (ii) kidney tissue comprising an elevated level of the polypeptide, where the polypeptide is a PCDH7 polypeptide, and (b) administering an immunosuppressant to the mammal. The mammal can be a human. The mammal can be identified as having the autoantibodies. The mammal can be identified as having the kidney tissue. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The method level of autoantibodies present within the mammal can be reduced by at least 50 percent following the administering step.

In another aspect, this document features methods for treating a mammal having membranous nephropathy. The methods can include, or consist essentially of, administering an immunosuppressant to a mammal identified as having (i) autoantibodies specific for a polypeptide or (ii) kidney tissue comprising an elevated level of the polypeptide, where the polypeptide is a PCDH7 polypeptide. The mammal can be a human. The mammal can be identified as having the autoantibodies. The mammal can be identified as having the kidney tissue. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

In another aspect, this document features methods for treating a mammal having membranous nephropathy and kidney tissue including an elevated level of a polypeptide, where the polypeptide is a PCDH7 polypeptide. The methods can include, or consist essentially of, administering an immunosuppressant to the mammal. The mammal can be a human. The mammal can have autoantibodies specific for the polypeptide. The mammal can be identified as having the kidney tissue. The kidney tissue can lack an elevated level of a NELL-1 polypeptide, can lack an elevated level of an EXT1 polypeptide, can lack an elevated level of an EXT2 polypeptide, and/or can lack an elevated level of a Semaphorin 3B polypeptide. The kidney tissue can lack an elevated level of a PLA2R polypeptide. The kidney tissue can lack an elevated level of a THSD7A polypeptide. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Discovery, screening, and validation cohorts of PCDH7-positive MN. FIG. 1A: In the discovery cohort, MS/MS was performed in 110 cases to look for novel proteins in PLA2R-negative MN. Six cases were positive for a unique protein PCDH7. IHC for PCDH7 was performed on the six cases and showed granular GBM staining for PCDH7. In the screening cohort, IHC was performed on 40 cases of PLA2R-negative MN, and two cases of PCDH7-positive MN were found. The findings were confirmed by MS/MS in both cases. FIG. 1B: In the validation cohorts, immunofluorescence microscopy (IF) for PCDH7 was performed in two cohorts. No positive cases were detected in the French cohort of 31 cases, while four positive cases were detected in the Belgian cohort of 38 cases.

FIGS. 2A-2D. Proteomic identification of PCDH7 in PLA2R-negative MN. Glomeruli were microdissected and analyzed using mass spectrometry as described in methods. FIG. 2A shows (i) two glomeruli marked for dissection and (ii) vacant space on slide following microdissection. FIG. 2B shows moderate spectral counts of PCDH7 in eight cases of PLA2R-negative MN. Numbers in italic font represent spectral counts of MS/MS matches to a respective protein. All eight cases show moderate total spectral counts for PCDH7 and immunoglobulins. Baseline spectral counts of PLA2R were detected in 6 of 8 cases. For comparison, the total spectral counts from six control cases (day 0 protocol transplant biopsies) are also shown. FIG. 2C: Representative sequence coverage map of PCDH7 from one case. The full-length amino acid sequence (SEQ ID NO:1) of a human PCDH7 polypeptide is presented. Amino acids highlighted in bold letters over yellow background are the amino acids detected. The majority of the amino acids detected are in the first 570 amino acids towards the N-terminus. Bold font indicates amino acids with artefactual chemical modification induced by mass spectrometry such as oxidation of methionine. FIG. 2D: An example of MS/MS spectra match to a sequence from PCDH7. Example MS/MS spectra of 504.93 m/z 3+ion matched to the protocadherin-7 peptide sequence LDETSGWLSVLHR (SEQ ID NO:2).

FIGS. 3A-3C. Immunohistochemical (IHC) and immunofluorescence microscopy for PCDH7-associated MN and control cases. FIG. 3A: PCDH7-associated MN (Mayo Clinic cohort). Eight cases show bright granular capillary wall staining for PCDH7 along the glomerular basement membranes. Each panel (panels A-G) shows three cases except bottom panel (only two cases). Top panel (from left to right): patient 1 (A), 2 (B), 3 (C); middle panel: patient 4 (D), 5 (E), 6 (F); bottom panel: patient 7 (G, low power 10x), patient 7 (G, 40x), patient 8 (H) (all 40x). FIG. 3B: PCDH7-associated MN (Validation cohort). Immunofluorescence microscopy shows (Panels A-C) bright capillary wall staining for PCDH7 of three cases of the Mayo cohort, (Panels D-G) bright capillary wall staining for PCDH7 in four cases of the Belgian validation cohort, and (Panels H-I) bright staining for PCDH7 in one patient from Mayo cohort (Panel H) and one patient from Belgian cohort (Panel I) (low power view, 20x). (Panel J) PCDH7 staining is negative in a case of PLA2R-negative MN. FIG. 3C: Control cases. PCDH7 staining is negative in a case of FSGS, lupus nephritis, diabetes, IgA nephropathy, PLA2R-negative MN, and a nephrectomy specimen.

FIGS. 4A-4G. Confocal immunofluorescence microscopy analysis: Detection of PCDH7 and IgG in glomerular immune deposits in PCDH7-associated MN. Glomeruli double-labeled with anti-PCDH7 (FIG. 4A) and anti-human IgG (FIG. 4B). FIG. 4C shows the merged image. (D-F) These images are enlarged images of the boxed areas in A, B and C, respectively. FIG. 4G: The graphs show quantitative analysis of the fluorescence recorded across sections of a representative capillary loop (indicated by arrows in FIG. 4F). Note the superimposition of the 2 signals, which indicates that subepithelial immune deposits contain PCDH7 (green) and IgG (red).

FIGS. 5A-5F. Biopsy finding of a representative case (patient 5) of PCDH7-associated MN: A. Light microscopy showing thickened glomerular basement membranes (periodic acid Schiff stain 40x), immunofluorescence microscopy showing (B) bright 3+ capillary wall staining for IgG, (C) mild 1+staining for C3, (D) bright 3+ staining for IgG1 along the capillary walls, (E) negative staining for IgG4, and (F) electron microscopy showing subepithelial electron dense deposits (7140x).

FIG. 6 is a schematic representation of PCDH7. The cadherin family is characterized by repeating motifs of extracellular cadherin (EC) domain. PCDH7 has a signal (S) peptide, seven EC domains, a single pass transmembrane domain (purple bar), and an intracellular (IC) cytoplasmic domain.

### DETAILED DESCRIPTION

This document provides methods and materials for identifying and/or treating mammals (e.g., humans) having membranous nephropathy (e.g., membranous nephropathy with an elevated level of a PCDH7 polypeptide in the GBM). For example, this document provides methods and materials for identifying a mammal (e.g., a human) having membranous nephropathy as having (a) autoantibodies specific for a PCDH7 polypeptide and/or (b) a GBM having an elevated level of a PCDH7 polypeptide.

Any appropriate mammal having membranous nephropathy can be identified as having (a) autoantibodies specific for a PCDH7 polypeptide and/or (b) kidney tissue (e.g., GBM) having an elevated level of a PCDH7 polypeptide. In some cases, a mammal having membranous nephropathy also can have one or more other diseases or disorders (e.g., a cancer such as a lung cancer or a breast cancer). Examples of mammals having membranous nephropathy that can be identified as having (a) autoantibodies specific for a PCDH7 polypeptide and/or (b) kidney tissue (e.g., GBM) having an elevated level of a PCDH7 polypeptide as described herein include, without limitation, primates (e.g., humans and monkeys), dogs, cats, horses, cows, pigs, sheep, rabbits, mice, and rats. For example, humans having membranous nephropathy can be identified as having (a) autoantibodies specific for a PCDH7 polypeptide and/or (b) kidney tissue such as GBM having an elevated level of a PCDH7 polypeptide as described herein. In some cases, a pediatric human less than 18 years of age (e.g., less than 15, 12, 10, 8, 6, 4, or 2 years of age) having membranous nephropathy can be identified as having (a) autoantibodies specific for a PCDH7 polypeptide and/or (b) kidney tissue such as GBM having an elevated level of a PCDH7 polypeptide as described herein.

Any appropriate method can be used to determine if a mammal (e.g., a human) has autoantibodies specific for a PCDH7 polypeptide. For example, immunological assays using a PCDH7 polypeptide (or a fragment thereof capable of binding to an anti-PCDH7 antibody) can be used to determine if a sample contains autoantibodies specific for a PCDH7 polypeptide. In some cases, an immobilized PCDH7 polypeptide (or an immobilized fragment thereof) can be used to capture an anti-PCDH7 autoantibody if present within a sample being tested, and an anti-Ig antibody (e.g., an anti-human IgG antibody when testing for human autoantibodies) can be used to determine whether or not autoantibodies were captured. In some cases, an anti-Ig antibody can be labeled (e.g., fluorescently or enzymatically labeled) to aid in detection. Any appropriate sample can be used to determine if a mammal (e.g., a human) has autoantibodies specific for a PCDH7 polypeptide. For example, blood samples (e.g., whole blood samples, serum samples, and plasma samples) or urine samples obtained from a mammal being tested can be used to determine if a mammal (e.g., a human) has autoantibodies specific for a PCDH7 polypeptide.

Any appropriate method can be used to determine if a mammal (e.g., a human) has kidney tissue (e.g., GBM) having an elevated level of a PCDH7 polypeptide. For example, immunological techniques such as immunohistochemistry (IHC) techniques, immunofluorescence (IF) techniques, or Western blot techniques can be used to determine if a mammal (e.g., a human) has kidney tissue (e.g., GBM) having an elevated level of a PCDH7 polypeptide. In some cases, a kidney tissue sample obtained from a mammal to be tested can be stained using an anti-PCDH7 antibody to determine if the mammal has kidney tissue (e.g., GBM) having an elevated level of PCDH7 polypeptides. Any appropriate sample can be used to determine if a mammal (e.g., a human) has kidney tissue (e.g., GBM) having an elevated level of a PCDH7 polypeptide. For example, kidney tissue biopsies can be obtained from a mammal (e.g., a human) being tested and used to determine if a mammal (e.g., a human) has kidney tissue (e.g., GBM) having a PCDH7 polypeptide.

The term “elevated level” as used herein with respect to a PCDH7 polypeptide level refers to a level of PCDH7 polypeptides present within kidney tissue (e.g., GBM) that is greater (e.g., at least 10, 25, 35, 45, 50, 55, 65, 75, 80, 90, or 100 percent greater) than the median level of PCDH7 polypeptides present within normal kidney tissue (e.g., a normal GBM) of comparable mammals not having membranous nephropathy.

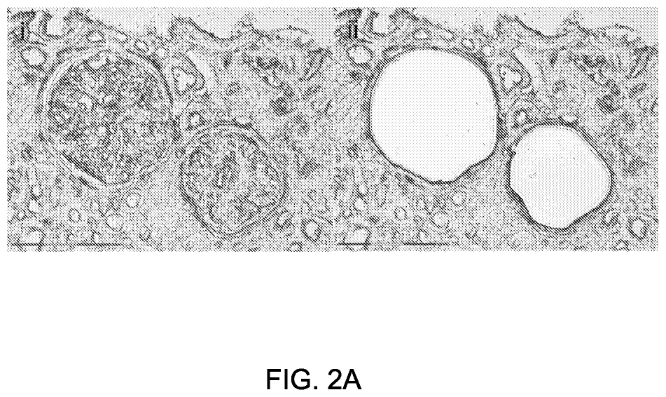

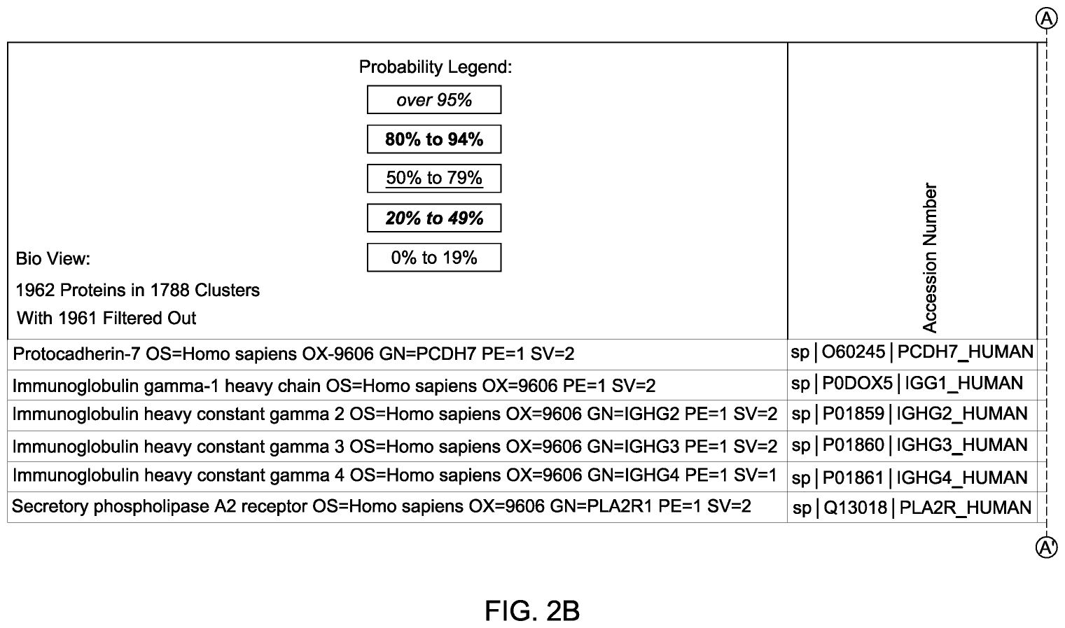

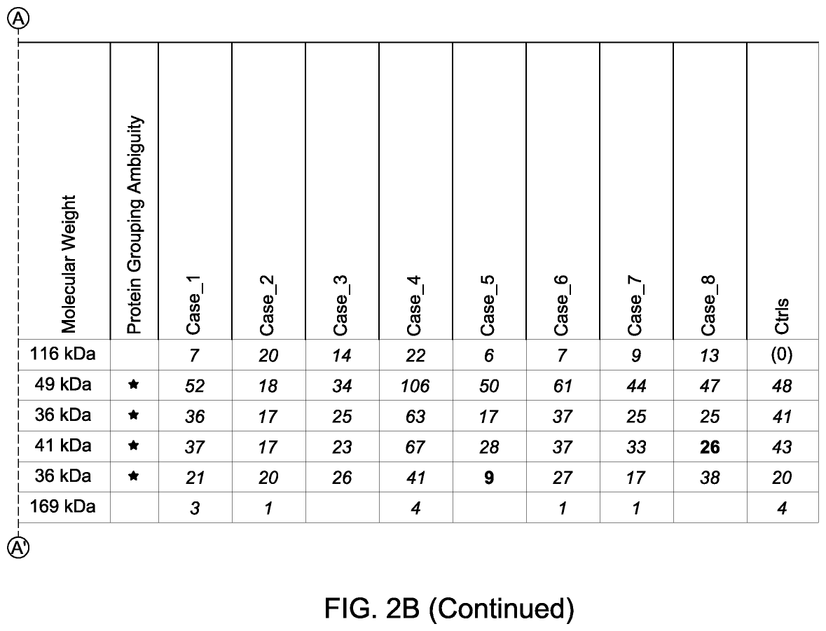

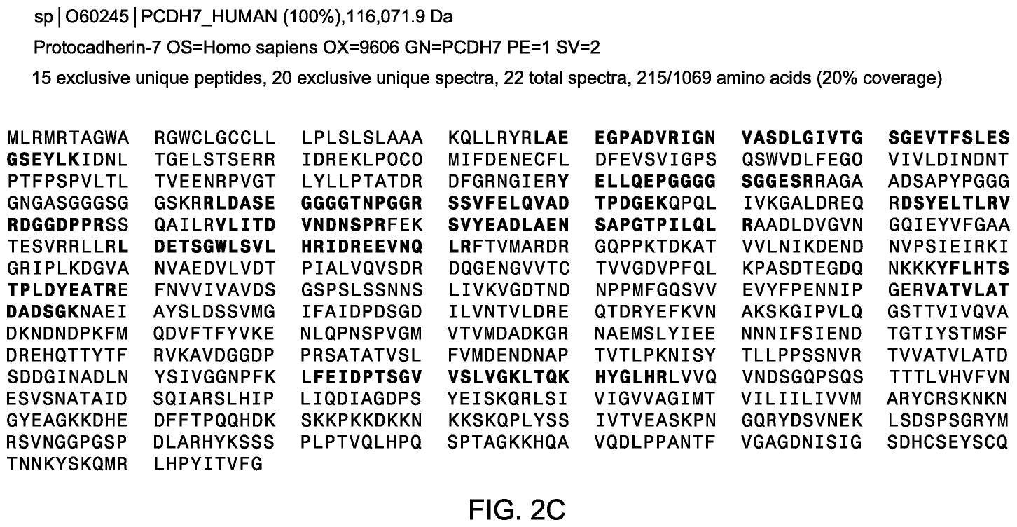

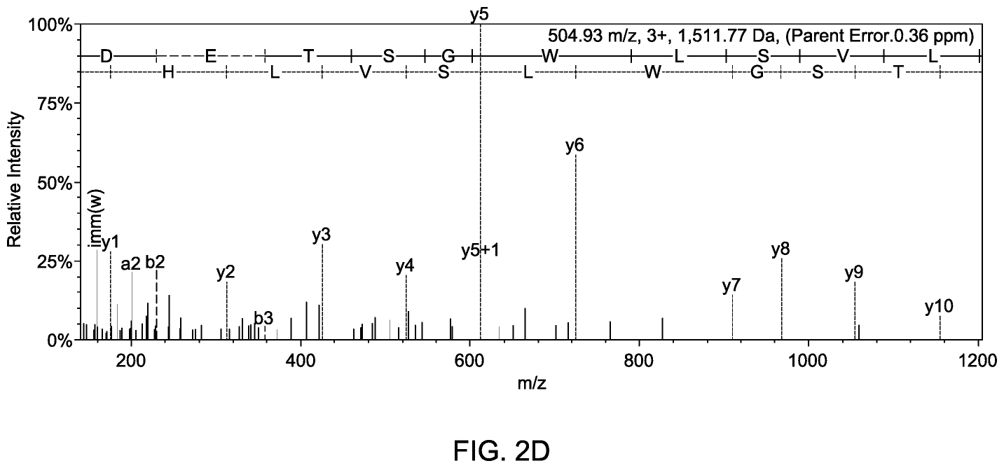

A PCDH7 polypeptide can include any appropriate amino acid sequence. An exemplary amino acid of a human PCDH7 polypeptide can include, without limitation, the amino acid sequence set forth in SEQ ID NO:1 (see, e.g., FIG. 2C). In some cases, the amino acid sequence of a PCDH7 polypeptide can have a sequence that deviates from the nucleotide sequence set forth in SEQ ID NO:1, sometimes referred to as a variant sequence. For example, a PCDH7 polypeptide can have an amino acid sequence that includes one or more modifications (e.g., deletions, insertions, and substitutions) to the amino acid sequence set forth in SEQ ID NO:1. For example, an amino acid sequence of a PCDH7 polypeptide can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the amino acid sequence set forth in SEQ ID NO:1. Percent sequence identity is calculated by determining the number of matched positions in aligned amino acid sequences, dividing the number of matched positions by the total number of aligned amino acids, respectively, and multiplying by 100. A matched position refers to a position in which identical amino acid occur at the same position in aligned sequences. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between an amino acid and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between an amino acid sequence and another sequence, the default parameters of the respective programs can be used. In some cases, a human PCDH7 polypeptide can have the amino acid sequence set forth in FIG. 2C.

Once a mammal (e.g., a human) having membranous nephropathy is identified as having autoantibodies specific for a PCDH7 polypeptide as described herein, the mammal can be classified as having membranous nephropathy that includes the presence of those autoantibodies (e.g., membranous nephropathy that includes the presence of anti-PCDH7 autoantibodies). In some cases, a mammal (e.g., a human) having membranous nephropathy that is identified as having autoantibodies specific for a PCDH7 polypeptide as described herein can be classified as having membranous nephropathy that includes kidney tissue having an elevated level of PCDH7 polypeptides.

Once a mammal (e.g., a human) having membranous nephropathy is identified as having kidney tissue (e.g., GBM) having an elevated level of a PCDH7 polypeptide as described herein, the mammal can be classified as having membranous nephropathy that includes the presence of that kidney tissue (e.g., membranous nephropathy that includes the presence of kidney tissue such as GBM having an elevated level of PCDH7 polypeptides). In some cases, a mammal (e.g., a human) having membranous nephropathy that is identified as having kidney tissue (e.g., GBM) having an elevated level of a PCDH7 polypeptide as described herein can be classified as having membranous nephropathy that includes autoantibodies specific for a PCDH7 polypeptide.

As described herein, this document also provides methods and materials for treating a mammal having membranous nephropathy. For example, a mammal (e.g., a human) having membranous nephropathy that is identified as having (a) autoantibodies specific for a PCDH7 polypeptide and/or (b) kidney tissue (e.g., GBM) having an elevated level of a PCDH7 polypeptide as described herein can be treated with one or more immunosuppressants. In some cases, a mammal (e.g., a human) having membranous nephropathy that is identified as having (a) autoantibodies specific for a PCDH7 polypeptide and/or (b) kidney tissue (e.g., GBM) having an elevated level of a PCDH7 polypeptide as described herein can be administered, or instructed to self-administer, one or more immunosuppressants to treat membranous nephropathy.

In some cases, a mammal (e.g., a human) having membranous nephropathy can be administered one or more immunosuppressants (e.g., anti-CD20 antibodies such as rituximab) to treat membranous nephropathy without attempting to determine if the mammal has autoantibodies specific for the following seven polypeptides: a PCDH7 polypeptide, a Semaphorin 3B polypeptide, a NELL-1 polypeptide, an EXT1 polypeptide, an EXT2 polypeptide, a PLA2R polypeptide, and a THSD7A polypeptide. In some cases, a mammal (e.g., a human) having membranous nephropathy can be administered one or more immunosuppressants (e.g., anti-CD20 antibodies such as rituximab) to treat membranous nephropathy without attempting to determine if the mammal has kidney tissue (e.g., GBM) having an elevated level of any of the following seven polypeptides: a PCDH7 polypeptide, a Semaphorin 3B polypeptide, a NELL-1 polypeptide, an EXT1 polypeptide, an EXT2 polypeptide, a PLA2R polypeptide, and a THSD7A polypeptide. In some cases, a mammal (e.g., a human) having membranous nephropathy can be administered one or more immunosuppressants (e.g., anti-CD20 antibodies such as rituximab) to treat membranous nephropathy without attempting to determine if the mammal has autoantibodies specific for those seven polypeptides and without attempting to determine if the mammal has kidney tissue (e.g., GBM) having an elevated level of any of those seven polypeptides. In some cases, a mammal (e.g., a human) having membranous nephropathy that is administered one or more immunosuppressants (e.g., anti-CD20 antibodies such as rituximab) to treat membranous nephropathy without attempting to determine the presence of such autoantibodies and such kidney tissue (e.g., GBM) can have autoantibodies specific for a PCDH7 polypeptide, can have autoantibodies specific for a Semaphorin 3B polypeptide, can have autoantibodies specific for a NELL-1 polypeptide, can have autoantibodies specific for an EXT1 polypeptide, can have autoantibodies specific for an EXT2 polypeptide, can have autoantibodies specific for a PLA2R polypeptide, or can have autoantibodies specific for a THSD7A polypeptide.

Any appropriate immunosuppressant can be administered to a mammal (e.g., a human that was identified as having (a) autoantibodies specific for a PCDH7 polypeptide and/or (b) kidney tissue (e.g., GBM) having an elevated level of a PCDH7 polypeptide as described herein) to treat membranous nephropathy. In some cases, an immunosuppressant used as described herein to treat membranous nephropathy can reduce inflammation and/or reduce B-cell autoantibody production within a mammal. Examples of immunosuppressants that can be used as described herein to treat membranous nephropathy include, without limitation, mycophenolate mofetil (e.g., Cellcept); steroids such as prednisone; B-cell inhibitors such as anti-CD20 antibodies (e.g., rituximab); calcineurin inhibitors such as cyclosporine and tacrolimus; and alkylating agents/chemotherapeutic drugs such as cyclophosphamide.

In some cases, two or more (e.g., two, three, four, five, six, or more) immunosuppressants can be administered to a mammal having membranous nephropathy (e.g., a human that was identified as having (a) autoantibodies specific for a PCDH7 polypeptide and/or (b) kidney tissue (e.g., GBM) having an elevated level of a PCDH7 polypeptide as described herein). For example, two immunosuppressants (e.g., prednisone and Cellcept) can be administered to a human having membranous nephropathy.

In some cases, one or more immunosuppressants can be administered to a mammal once or multiple times over a period of time ranging from days to months. In some cases, one or more immunosuppressive drugs can be given to achieve remission of membranous nephropathy, and then given during follow up periods to prevent relapse of the membranous nephropathy. In some cases, one or more immunosuppressants can be formulated into a pharmaceutically acceptable composition for administration to a mammal (e.g., a human) having membranous nephropathy to reduce inflammation and/or to reduce B-cell autoantibody production within that mammal. For example, a therapeutically effective amount of an immunosuppressant can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more immunosuppressants can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

In some cases, a pharmaceutically acceptable composition including one or more immunosuppressants can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the nephropathy, the route of administration, the age and general health condition of the subject, excipient usage, the possibility of co-usage with other therapeutic treatments, and the judgment of the treating physician.

An effective amount of a composition containing one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, an effective amount of rituximab to treat membranous nephropathy as described herein can be from about 500 mg to about 1.5 g (e.g., from about 500 mg to about 1.25 g, from about 500 mg to about 1.0 g, from about 500 mg to about 750 mg, from about 750 mg to about 1.5 g, from about 1 g to about 1.5 g, or from about 1.25 g to about 1.5 g) administered IV about two weeks apart. In some cases, an effective amount of rituximab to treat membranous nephropathy as described herein can be from about 200 mg/m² to about 500 mg/m² (e.g., from about 200 mg/m² to about 450 mg/m², from about 200 mg/m² to about 400 mg/m², from about 200 mg/m² to about 375 mg/m², from about 250 mg/m² to about 500 mg/m², from about 300 mg/m² to about 500 mg/m², from about 350 mg/m² to about 500 mg/m², or from about 350 mg/m² to about 400 mg/m²) administered weekly for about four weeks. If a particular mammal fails to respond to a particular amount, then the amount of an immunosuppressant can be increased by, for example, two fold. After receiving this higher amount, the mammal can be monitored for both responsiveness to the treatment and toxicity symptoms, and adjustments made accordingly. For example, levels of anti-PCDH7 autoantibodies present within the mammal (e.g., within the blood of the mammal) can be monitored by an appropriate method (e.g., ELISA, laser microdissection, and mass spectrometry). In some cases, the effective amount of a composition containing one or more immunosuppressants can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition can require an increase or decrease in the actual effective amount administered.

The frequency of administration of one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, the frequency of administration of an immunosuppressant can be from about once a day to about once a month (e.g., from about once a week to about once every other week). The frequency of administration of one or more immunosuppressants can remain constant or can be variable during the duration of treatment. A course of treatment with a composition containing one or more immunosuppressants can include rest periods. For example, a composition containing one or more immunosuppressants can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more immunosuppressants can be any duration that reduces inflammation or B-cell autoantibody production (e.g., B-cell antibody production inhibition or reduction in the number of B-cells) within a mammal having membranous nephropathy without producing significant toxicity to the mammal. In some cases, the effective duration can vary from several days to several months. In general, the effective duration for administering a composition containing one or more immunosuppressants to treat membranous nephropathy can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective duration for administering a composition containing one or more immunosuppressants to treat membranous nephropathy can be for as long as the mammal is alive. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to membranous nephropathy can be monitored. Any appropriate method can be used to determine whether or not membranous nephropathy is being treated. For example, immunological techniques (e.g., ELISA, laser microdissection, and mass spectrometry) can be performed to determine if the level of autoantibodies (e.g., anti-PCDH7 autoantibodies, anti-Semaphorin 3B autoantibodies, anti-NELL-1 autoantibodies, anti-EXT1 autoantibodies, anti-EXT2 autoantibodies, anti-PLA2R autoantibodies, and/or anti-THSD7A autoantibodies) present within a mammal being treated as described herein is reduced following the administration of one or more immunosuppressants. Remission and relapse of the disease can be monitored by testing for one or more markers for membranous nephropathy. In some cases, remission can be ascertained by detecting the disappearance or reduction of autoantibodies to PCDH7, Semaphorin 3B, NELL-1, THSD7A, PLA2R, EXT1, and/or EXT2 in the sera. In some cases, relapse of membranous nephropathy can be ascertained by a reappearance or elevation of autoantibodies to PCDH7, Semaphorin 3B, NELL-1, THSD7A, PLA2R, EXT1, and/or EXT2 in the sera.

The invention will be further described in the following examples, which do not limit the scope of the invention described in the claims.

### EXAMPLES

### Example 1

### Protocadherin-7 (PCDH7)-associated Membranous Nephropathy

The results provided herein demonstrate that PCDH7 polypeptides are present in a subset of PLA2R-, THSD7A-, EXT1-, EXT2-, NELL1-, and Semaphorin 3B-negative MN patients representing a distinct type of primary MN. Accordingly, the presence of PCDH7 polypeptides in a sample obtained from a patient (e.g., a human) can be used to identify the mammal as having PCDH7-positive MN.

### Patients and Sample Collection

Biopsies received in the Renal Pathology Laboratory, Department of Laboratory Medicine and Pathology, Mayo Clinic, for diagnosis and interpretation were evaluated. The diagnosis of MN was confirmed by light microscopy, immunofluorescence microscopy including PLA2R studies, and electron microscopy. The clinical information was obtained from the accompanying charts.

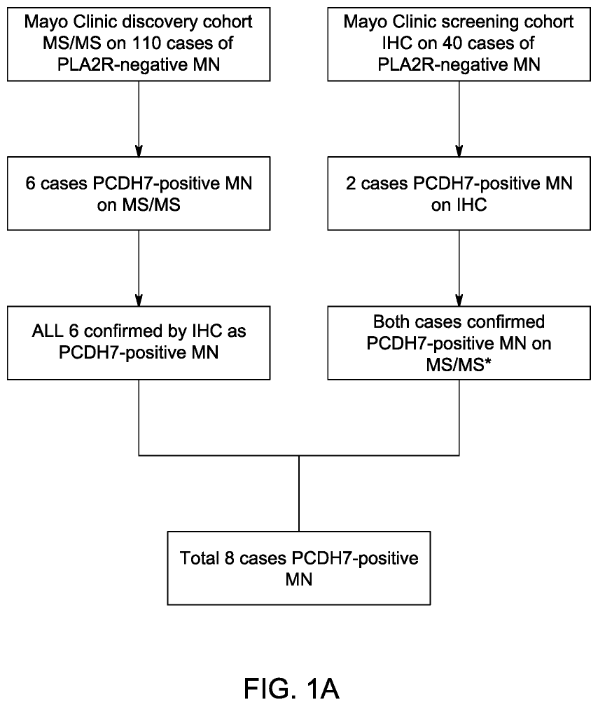

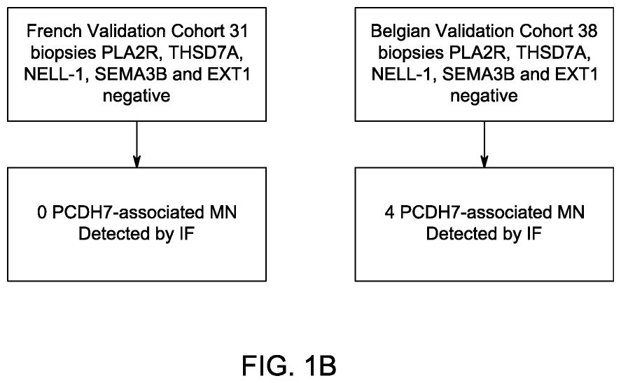

For detection of involved proteins, MS/MS was performed in 110 cases of PLA2R-negative cases that included the cases used for identification of EXT1/EXT2, NELL1, and Sema3B (Sethi et al., J. Am. Soc. Nephrol., 30:1123-1136 (2019); Sethi et al., Kidney International, 97:163-174 (2020); and Sethi et al., Kidney International, PMID32534052 https://doi.org/10.1016/j.kint.2020.05.030 (2020) “Sethi et al., Kidney International (2020)”). Six cases of PCDH7-positive MN were detected by MS/MS. All cases were negative for spectral counts for THSD7A, EXT1/EXT2, NELL1, and Sema3B, while baseline spectral counts for PLA2R were present. All six cases were then stained for PCDH7 using immunohistochemistry (IHC). Another 40 cases of PLA2R-negative MN were then screened for PCDH7 by IHC. An additional two cases of PCDH7 were detected (FIG. 1). MS/MS was performed in these two IHC positive cases to confirm IHC findings and detect PCDH7. The two cases were negative for THSD7A, EXT1/EXT2, NELL1, and Sema3B, while baseline spectral counts for PLA2R were present. For control cases, MS/MS was performed on 116 cases that included 15 cases of time 0 kidney transplant biopsies, 17 cases of minimal change disease, 44 cases of focal segmental glomerulosclerosis, 7 cases of diabetic glomerulosclerosis, 5 cases of IgA nephropathy, and 28 cases of PLA2R-associated MN. The PLA2R-negative MN and control cases were the same cases that were used for MS/MS studies in the detection of EXT1/EXT2, NELL1, and Sema3B (Sethi et al., J. Am. Soc. Nephrol., 30:1123-1136 (2019); Sethi et al., Kidney International, 97:163-174 (2020); and Sethi et al., Kidney International, (2020)).

For control IHC, paraffin embedded material was used from 24 biopsies that included: 4 cases of focal segmental glomerulosclerosis, 5 cases of IgA nephropathy, 1 case of lupus nephritis, 6 cases of diabetes, 6 cases of PLA2R-associated MN, and 2 cases of normal kidney tissue from nephrectomy specimens for tumors.

Two validation cohorts were screened by immunofluorescence microscopy (IF) for PCDH7-associated MN: (1) the French cohort that included 31 biopsies, and (2) a second validation cohort consisting of 38 biopsies from the UCLouvain Kidney Disease Network, in Belgium (Hanset et al., Am. J Kid. Dis., 2020 Jul 12;50272-6386(20)30795-2. doi: 10.1053/j.ajkd.2020.04.013). Validation studies by IF were performed at Tenon Hopital, Paris. All biopsies of the validation cohort were PLA2R, THSD7A, NELL1, EXT1, and Sema3B negative. In addition, 3 cases of PCDH7-associated MN of the Mayo Clinic cohort also were confirmed by IF along with staining of the validation cohort cases.

### Protein Identification by Laser Capture Microdissection, Trypsin Digestion, Nano-LC Orbitrap Tandem Mass Spectrometry (MS/MS)

For each case, 10-micron thick formalin-fixed paraffin sections (FFPE) were obtained and mounted on a special PEN membrane laser microdissection slide. Using a Zeiss Palm Microbean microscope, the glomeruli were microdissected to reach approximately 250-550000 μM² per case. Resulting FFPE fragments were digested with trypsin and collected for MS/MS analysis. The trypsin digested peptides were identified by nano-flow liquid chromatography electrospray tandem MS/MS (nanoLC-ESI-MS/MS) using a Thermo Scientific Q-Exactive Mass Spectrometer (Thermo Fisher Scientific, Bremen, Germany) coupled to a Thermo Ultimate 3000 RSLCnano HPLC system. All MS/MS samples were analyzed using Mascot and X! Tandem set up to search a Swissprot human database. Scaffold (version 4.8.3, Proteome Software Inc., Portland, Oreg.) was used to validate MS/MS based peptide and protein identifications. Peptide identifications were accepted at greater than 95.0% probability by the Scaffold Local FDR algorithm with protein identifications requiring a 2 peptide minimum and a 95% probability using Protein Prophet (Nesvizhskii et al., Anal. Chem., 75:4646-4658 (2003)). The areas dissected for each PCDH7 positive case was as follows: patient 1-84192 μM², patient 2-553651 μM², patient 3-244970 μM², patient 4-529608 μM², patient 5-444269 μM², patient 6-529725 μM², patient 7-519303 μM², and patient 8-91894 μM².

### Immunohistochemical Staining for PCDH7

Tissue sectioning and IHC staining was performed using the Leica Bond RX stainer (Leica). FFPE tissues were sectioned at 5 microns, and IHC staining was performed on-line. Slides for PCDH7 stain were retrieved for 20 minutes using Epitope Retrieval 2 (EDTA; Leica). The PCDH7 Mouse Monoclonal (Clone OT12G6; Abcam) was diluted to 1:400 in Background Reducing Diluent (Dako) and incubated for 15 minutes. The detection system used was Polymer Refine Detection System (Leica). This system includes the hydrogen peroxidase block, post primary and polymer reagent, DAB, and Hematoxylin.

Immunostaining visualization was achieved by incubating slides 10 minutes in DAB and DAB buffer (1:19 mixture) from the Bond Polymer Refine Detection System. To this point, slides were rinsed between steps with 1X Bond Wash Buffer (Leica). Slides were counterstained for five minutes using Schmidt hematoxylin and molecular biology grade water (1:1 mixture), followed by several rinses in 1X Bond wash buffer and distilled water. Once the immunochemistry process was completed, slides were removed from the stainer and rinsed in tap water for five minutes. Slides were dehydrated in increasing concentrations of ethyl alcohol and cleared in 3 changes of xylene prior to permanent cover slipping in xylene-based medium.

### IF Staining and Confocal Analysis

IF staining was performed on FFPE sections retrieved for 30 minutes using target retrieval solution high pH (Dako) in pressure cooker equipment (Bio SB, Santa Barbara, Calif.). The PCDH7 primary antibody (mouse monoclonal to PCDH7, Clone OT12G6; Abcam) was diluted to 1:200 in blocking solution (2% calf fetal serum and 2% normal goat serum) and incubated overnight at 4° C. with retrieved biopsy sections. Next, the slides were incubated with goat Alexa 488-conjugated Fab IgG anti-mouse antibodies (dilution 1:400, Life Technologies) as secondary antibody. Anti-human IgG Alexa Fluor 647 rabbit monoclonal antibody (dilution 1:50 Abcam) was then reacted with the retrieved tissue as described above. Finally, slides were mounted in mounted medium (Thermo Fisher Scientific) and covered with LDS2460EP cover glass slides. Co-localization of PCDH7 and IgG along the glomerular basement membrane was examined by confocal microscopy using a Leica TCS-SP2 and analyzed with Leica Confocal Software (version 2.61; Leica, Wetzlar, Germany).

### Results

### Laser Dissection and Mass Spectrometry (MS/MS) Detection of PCDH7 in PLA2R-Negative MN Biopsies

A unique protein, PCDH7, was detected by MS/MS in the glomeruli of 8 cases of MN (FIG. 2). The counts ranged from 6 to 22 with an average total spectral count of 12.3 (SD ±6.1). The average spectral counts of PCDH7 were lower than PLA2R (86.1, S.D 27.5), EXT1/EXT2 (EXT1 65.3, S.D±34.6; EXT2 83.4, S.D±38.4), NELL-1 (63.1, S.D 21.6), and Sema3B (23.7±16.5) in PLA2R-, EXT1/EXT2-, NELL-1-, and Sema3B-associated MN, respectively (Sethi et al., J. Am. Soc. Nephrol., 30:1123-1136 (2019); Sethi et al., Kidney International, 97:163-174 (2020); and Sethi et al., Kidney International, (2020)). However, the finding of PCDH7 was unique in this subset of PLA2R-negative MN, and all control cases including 15 time 0 transplant biopsies, 73 other glomerulopathies, and 28 PLA2R-positive MN cases were negative for PCDH7.

Representative laser microdissection of a case of PCDH7 is shown in FIG. 2A. The spectral counts of all 8 PCDH7-positive cases along with a representative sequence coverage map of PCDH7 are shown in FIGS. 2B-C. The MS/MS spectra match from one case is shown in FIG. 2D. None of the PCDH7-positive cases revealed any spectral counts for EXT1/EXT2, THSD7A, NELL-1, or Sema3B while baseline PLA2R counts were detected in 5 of the 8 cases but the counts were much lower than PCDH7 and similar to the baseline PLA2R counts seen in EXT1/EXT2, NELL-1, and Sema3B-associated MN.

All four classes of Ig were detected in PCDH7-associated MN, with average spectral counts of IgG1 46.5 (S.D±30.6), IgG2 30.6 (S.D±15.0), IgG3 33.5 (S.D±15.1), and IgG4 24.5 (S.D±10.6).

### Immunohistochemical Staining for PCDH7 in PLA2R-Negative Biopsies

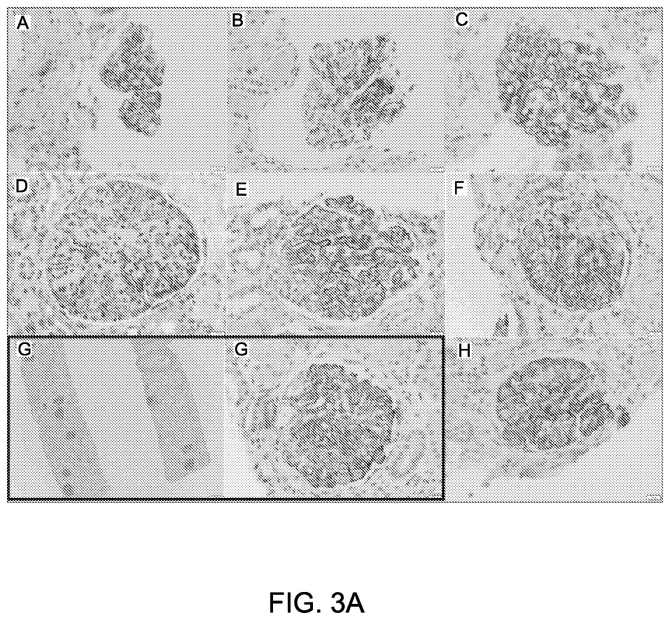

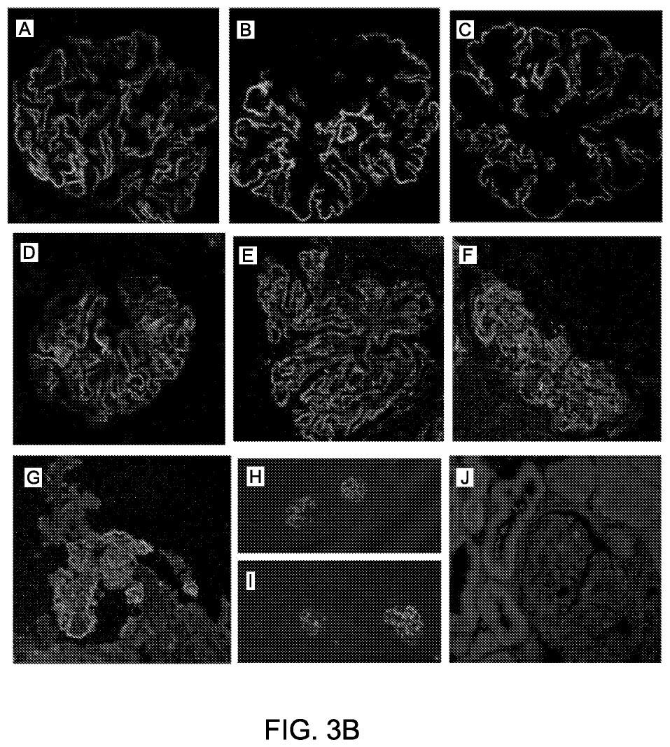

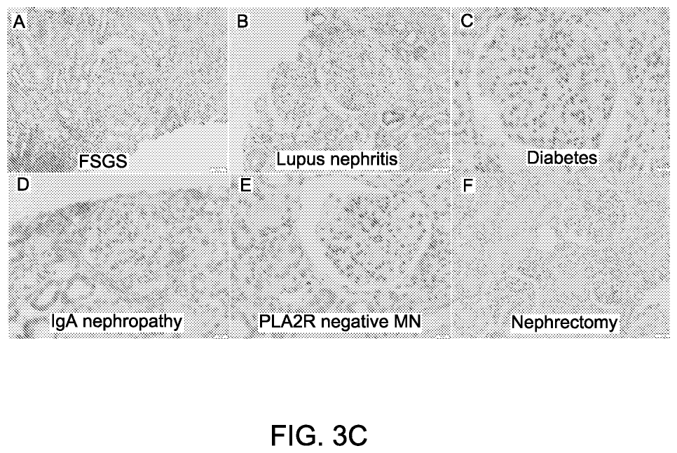

Eight cases were positive for PCDH7. All 8 positive cases showed bright (2-3+/3) granular staining for PCDH7 along the GBM (FIG. 3A). The positive PCDH7 granular staining mirrored the granular IgG along the GBM seen in each case. There was no significant mesangial staining. There was no staining along the Bowman's capsule. In addition, 3 positive cases of PCDH7 were also stained by IF along with the validation cohorts and showed bright PCDH7 staining along the GBM (FIG. 3B). All control cases were negative for PCDH7 staining along the GBM. Representative negative staining for PCDH7 in FSGS, lupus nephritis, diabetes, IgA nephropathy, PLA2R-negtive MN, and a nephrectomy specimen is shown in FIG. 3C.

### Validation Cohorts

Two validation cohorts were screened to detect additional cases of PCDH7-associated MN. A total of 69 biopsies were screened of which 4 (5.8%) patients were positive for PCDH7.

French cohort: 31 biopsies of PLA2R-, THSD7A-, NELL-1-, EXT1-, and Sema3B-negative MN were screened for PCDH7. All 31 cases were negative for PCDH7.

Belgian cohort: 38 biopsies of PLA2R-, THSD7A-, NELL-1-, EXT1-, and Sema3B-negative MN were screened for PCDH7. Four (10.5%) cases were positive for PCDH7 (FIG. 3B).

### Confocal Microscopy

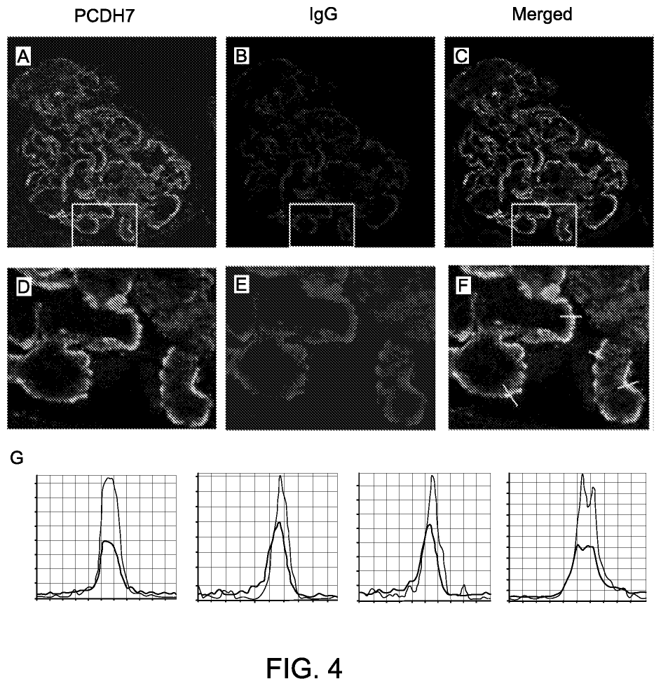

Confocal immunofluorescence microscopy was performed to show that the PCDH7 and IgG co-localized along the GBM (FIG. 4). Superimposition of the two signals (yellow, FIGS. 4C, F) and laser quantitative analysis (FIG. 4G) confirmed the co-localization of PCDH7 and IgG further corroborating that the subepithelial deposits contain both PCDH7 and IgG.

Clinical and kidney biopsy findings of PCDH7-associated MN (Table 1) Mayo Clinic cohort: Eight cases of PCDH7-associated MN (patients 1-8) were identified. There were 7 (87.5%) male patients and 1 (12.5%) female patient. The age at presentation was 58.6 (SD±10.4) years. The serum creatinine and proteinuria at presentation was 1.3 mg/dL (SD±0.3) and 4.3 g/24 hours (SD±2.8), respectively. Patient 4 had overlap syndrome of Sjogren's syndrome/lupus with positive ANA, SSA, and SSB titers, but negative anti-dsDNA titers. Patient 5 had positive ANA titers, but other markers for lupus including dsDNA titers were negative. One patient had a prostate carcinoma, and one had an oncocytic nodule. Two patients had a recent diagnosis (within last 6 months), thus with almost no follow up. One patient was lost to follow up. Of the remaining 5 patients, the proteinuria decreased to 1-1.3 g/24 hours in 4 patients whereas one patient continued to have proteinuria of 6 g/24 hours at last follow up. Treatment of the 5 patients were as follows: Patients 1 and 6 were managed conservatively, patient 2 received anti-CD20 treatment, patient 4 was treated with prednisone and mycophenolate mofetil, and patient 8 was treated with ACTHAR gel.

The kidney biopsy of all cases of PCDH7-associated MN showed the characteristic findings of thickened GBM on light microscopy, bright IgG staining along the capillary wall on immunofluorescence microscopy, and subepithelial deposits on electron microscopy.

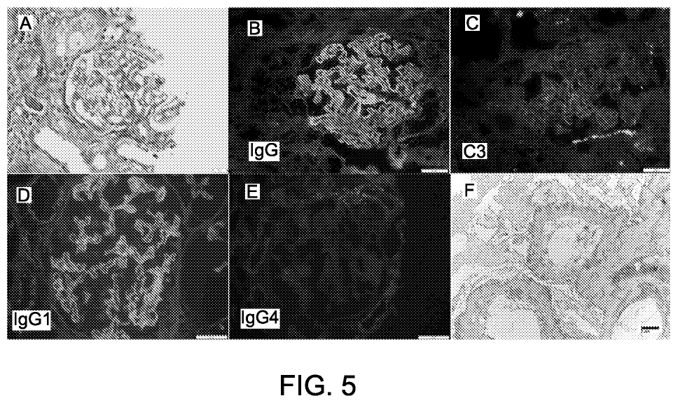

Overall, an average of 24.6 (SD±13.7) glomeruli were present of which 6.5 (SD±8.4) were globally sclerosed. Immunofluorescence microscopy showed bright staining for IgG (2-3+/3) in all cases indicating antibody deposition along the GBM. Interestingly, C3 was trace or absent in 6 of the 8 cases, and the remaining two cases showed only 1+ C3 staining. Three cases showed trace-1+ staining for C1q. All cases showed staining for kappa (2-3+/3) and lambda (2-3+/3) light chains. IgG subtype staining done in six cases showed IgG1 in three cases, IgG2 in one case, IgG3 in three cases, and IgG4 in five cases. Immunofluorescence staining for PLA2R was negative in all cases. Electron microscopy showed subepithelial deposits in all cases. Subendothelial and mesangial deposits were not present. Tubuloreticular inclusions were present in two cases. A representative case is shown in FIG. 5.

Belgian cohort: Four cases were identified in the Belgian cohort. There were no cases in the French cohort. The mean age at presentation was 66 years (SD±10.4). There was one male patient and three female patients. Mean (SD) serum creatinine at diagnosis was 1.8 mg/dL (1.1), and urinary protein excretion was 14.2 g/24 hours (8.2). In patient 9, the diagnosis of MN was concurrent with that of primary Sjogren syndrome. The patient achieved partial remission (UPCR 0.9 g/g, eGFR 72 mL/min/1.73m²) after treatment with rituximab and corticosteroids, followed by maintenance therapy with mycophenolate mofetil. Patient 11 had a history of sarcoidosis. Partial remission (UPCR 0.7 g/g) was achieved after cyclophosphamide and corticosteroids followed by azathioprine for maintenance therapy. Patients 10 and 12 had no associated conditions. Patient 10 spontaneously progressed to partial remission (UPCR 1.0 g/g), and no follow-up information was available for patient 12. Two out of the four cases (i.e. those not associated with systemic conditions) showed no glomerular C3 deposits. IgG subtypes done in three cases showed predominant IgG3 in one case and IgG4 in two cases.

TABLE 1
Clinical and pathologic findings in PCDH7-associated MN (Mayo
Clinic cohort patients 1-8, Belgian cohort patients 9-12).

| | | | Urinary | Serum | Urinary | Serum | Sclerosed/ | | | | |
|---|---|---|---|---|---|---|---|---|---|---|---|
| | Age | | protein | Cr | protein | Cr | Total | IFTA | | | |
| Patient | (years) | Sex | gm/24 hrs | mg/dL | gm/24 hrs * | mg/dL* | glomeruli | % | IF | IgG | EM |
| 1 | 73 | M | 3.2 | 1.1 | 1.3 | 1.2 | 0/12 | 0 | IgG 3+, | IgG1 2+ | II |
| | | | | | (36 m) | (36 m) | | | Clq 1+ | IgG3 1+ | |
| | | | | | | | | | C3 +/− | IgG4 1+ | |
| 2 | 66 | M | 9.6 | 1.3 | 6 | 1.0 | 3/16 | 10 | IgG 2+ | IgG1 1+ | II, TRI |
| | | | | | (36 m) | (36 m) | | | | IgG4 2+ | rare |
| 3 | 68 | M | NA | 1.1 | NA | NA | 2/18 | 10 | IgG 2+ | IgG4 2+ | II |
| 4 | 59 | F | 3 | 1.1 | 1.3 | 1.5 | 21/51 | 25 | IgG 2+, | IgG1 1+ | II, TRI |
| | | | | | (6 m) | (6) | | | C3 +/− | IgG2 2+ | |
| | | | | | | | | | to 1+ | IgG3 2+ | |
| | | | | | | | | | | IgG4 3+ | |
| 5 | 61 | M | 7 | 1.9 | Recent | Recent | 3/11 | 10 | IgG 3+, | IgG1 2+ | II |
| | | | | | diagnosis | Diagnosis | | | C1q 1+ | IgG3 2+ | |
| | | | | | (<6 m) | (<6 m) | | | C3 1+ | | |
| 6 | 38 | M | 3 | 1 | 1 | 0.9 | 2/33 | 0 | IgG 1+ | No | II |
| | | | | | (6 m) | (6 m) | | | C1q/ | glomeruli | |
| | | | | | | | | | C3+/− | | |
| 7 | 37 | M | 1.4 | 1.76 | Recent | Recent | 2/34 | 25 | IgG3+ | IgG4 1+ | III-IV |
| | | | | | diagnosis | Diagnosis | | | C3 +/− | | |
| | | | | | (<6 m) | (<6 m) | | | | | |
| 8 | 67 | M | 4.3 | 1.2 | 1.3 | 1.4 | 19/22 | 30 | IgG3+ | Not done | III |
| | | | | | (24 m) | (24 m) | | | C3 1+ | | |
| 9 | 64 | F | 8.4 | 1.2 | 0.9 | 0.9 | 3/15 | <25 | IgG3+ | IgG3 3+ | I |
| | | | | | (20 m) | (20 m) | | | C3 3+ | IgG2 2+ | |
| | | | | | | | | | C1q 1+ | IgG1/4 | |
| | | | | | | | | | | trace | |
| 10 | 61 | M | 3.9 | 1.0 | 1.0 | 1.5 | 2/17 | <25 | IgG 3+ | IgG4 2+ | I |
| | | | | | (19 m) | (19 m) | | | | IgG2 1+ | |
| 11 | 66 | F | 23.3 | 3.8 | 0.7 | 1.8 | 1/6 | <25 | IgG 3+ | IgG4 2+ | I |
| | | | | | (23 m) | (23 m) | | | IgA 2+, | IgG2 2+ | |
| | | | | | | | | | C3 3+ | | |
| 12 | 72 | F | 21 | 1.3 | NA | NA | 2/5 | <25 | IgG1+ | Not done | I |

Follow-up in months, M: male, F: Female, Cr: creatinine, IFTA: interstitial fibrosis and tubular atrophy, IF: immunofluorescence microscopy, EM: electron microscopy, and TRI: tubuloreticular inclusion.

Complement in PCDH7-associated MN

Immunofluorescence microscopy showed minimal to 1+staining for C3 in all Mayo Clinic cases of PCDH7-associated MN. This is in contrast to PLA2R-, EXT1/EXT2-, NELL-1-, and Sema3B-associated MN which show bright staining for C3 (Sethi et al., J. Am. Soc. Nephrol., 30:1123-1136 (2019); Sethi et al., Kidney International, 97:163-174 (2020); and Sethi et al., Kidney International, (2020)). The MS/MS complement profile of PCDH7-associated MN was analyzed, and the findings were compared with PLA2R- and EXT1/EXT2-associated MN (Ravindran et al., Kidney International Reports, 5:618-626 (2020)). C3, C4, C5, and C9 spectral counts were lower in PCDH7-associated MN in comparison to PLA2R-associated MN. The average spectral counts of C3 was 133.4 (SD ±55.8), C4A was 71 (SD±15.6), C4B was 77.5 (SD±23.6), C5 was 19.8 (SD±19.4), and C9 was 25.3 (SD±18.5), respectively, in PCDH7-associated MN. Compared to PCDH7-associated MN, the average count of C3 was 413 (S.D±136, P=0.004), C4A was 174 (S.D±59, P=0.005), C4B was 177 (S.D±63, P=0.01), C5 was 56 (S.D±26, P=0.04), and C9 was 79 (S.D±32, P=0.01), respectively, in PLA2R-associated MN (Ravindran et al., Kidney International Reports, 5:618-626 (2020)).

### Discussion

PLA2R and THSD7A account for approximately 70% and 1-5% of the antigens of primary MN, respectively. The last few years have seen the discovery of new antigens in MN including antigens EXT1/EXT2, NELL-1, and Sema3B. In all, when PLA2R, THSD7A, NELL-1, Sema3B, and EXT1/EXT2 are added up, they account for 80-90% of the antigens in MN. In order to find the antigens in the remaining 10-20% cases of MN, the studies utilizing the technique of laser microdissection and mass spectrometry described herein were carried out.

The starting point of the laser microdissection and MS/MS studies was evaluation of MN kidney biopsies that were PLA2R-negative on immunofluorescence studies. As described herein, a unique protein was detected in the PLA2R-negative MN cohort: a 116 KDa protein called Protocadherin 7 (PCDH7). In terms of occurrence, PCDH7 is the third most common novel protein after EXT1/EXT2 and NELL-1 in the MS/MS cohort of PLA2R-negative MN.

The common theme in all PCDH7-positive cases was the relatively low PCDH7 spectral counts. However, the counts were high enough for 100% identification of the protein. The protein appeared unique in that it was not detected in any of the PLA2R-negative cases or in any of the control cases. A closer look at the structure of PCDH7 revealed that it is a heavily glycosylated glycoprotein, in particular amino acids 689-845 (UniProtKB Record Number 060245).

During the process of MS/MS, glycosylation can interfere with trypsin's access and inhibit its binding and cleavage of the arginine and lysine residues in the glycosylated region. Thus, if peptides with glycosylation are generated and are present in the sample, the higher energy collisional dissociated (HCD) MS/MS fragmentation process preferentially fragments the glycan chains at the expense of the peptide bonds. This gives a MS/MS spectra that are, mostly if not all, glycan fragments and not spectra of the amino acids. Thus, a closer study of representative sequence coverage of PCDH7 revealed that mostly peptide sequences that are most heavily glycated (amino acids 689-845) are not represented in the coverage sequence (lower half of amino acids in FIG. 2C) and likely account for the low spectral counts on MS/MS. It should be pointed out that even lower spectral counts were acceptable for confirmation of various amyloid subtypes as long as the protein is unique and fits the criteria of amyloid diagnosis (the amyloid signature) (Sethi et al., Clin. J. Am. Soc. Nephrol., 5:2180-2187 (2010); Sethi et al., Curr. Opin. Nephrol. Hyperten., 22:273-280 (2013); and Vrana et al., Blood, 114:4957-4959 (2009)). Finally, IHC and IF studies showed bright staining for PCDH7 along the GBM, thus further suggesting that the low spectral counts in PCDH7-associated MN do not indicate decreased expression of the protein.

IHC showed the typical granular staining for PCDH7 along the GBM mirroring the IgG in the PCDH7-positive cases. IHC was negative for GBM staining of PCDH7 in the control cases and in the remaining PLA2R-negative and PLA2R-positive cases. Confocal immunofluorescence microscopy confirmed the co-localization of IgG and PCDH7 indicating that PCDH7 is the likely target of IgG. Interestingly, there was focal PCDH7 staining along the tubular basement membranes and in the tubular epithelial cells. This is in keeping with previously known expression of PCDH7 in the kidney. The normal glomeruli show no/weak glomerular staining for PCDH7 while the staining is of moderate intensity in the tubules (https://www.proteinatlas.org/ENSG00000169851-PCDH7/tissue/kidney).

A feature seen in 10 of the 12 biopsies of PCDH7-associated MN was that the complement activation was minimal. Kidney biopsy showed none/trace/1+ staining for either C1q or C3. The minimal C1q/C3 was seen in all 8 biopsies of Mayo Clinic cohort and was confirmed by mass spectrometric analysis of complement profile of PCDH7-positive MN. The lack of complement accumulation in PCDH7-positive MN is quite unlike that seen in other forms of MN including PLA2R-, NELL-1-, and EXT1/EXT2-associated MN. Thus, minimal complement deposition in cases of MN may be an identifying feature of PCDH7-positive MN. It is possible that the heavily glycosylated PCDH7 immune-complexes do not activate the complement pathways.

8 (5.3%) PCDH7-associated MN cases were identified out 150 cases in the Mayo Clinic cohort, and 4 (5.8%) PCDH7-associated MN cases were identified out of 69 cases of the combined Belgian and French cohort. Both the Mayo Clinic and Belgian cohort cases were all PLA2R-, THSD7A-, EXT1-, NELL-1-, and Sema3B-negative. Taken together, the findings suggest that the prevalence of PCDH7-associated MN is approximately 1.5-3%. Finally, 4 of the 12 patients had secondary diseases associated with MN: 2 (16.6%) patients had Sjogren's syndrome/lupus, one (8.3%) had sarcoidosis, and one (8.3%) had a malignancy. This is another example where a protein can be associated with both the primary and so-called secondary MN arguing for a classification based on the serology and antigen detected.

Since this was a retrospective study, access was available for only one serum sample of a PCDH7-associated MN patient. Western blot analysis for antibodies to PCDH7 was negative in this patient. This patient (patient #1) was in remission at the time of serum collection. Analysis of serum from PCDH7-associated MN patients with active disease are expected to contain auto-antibodies to PCDH7.

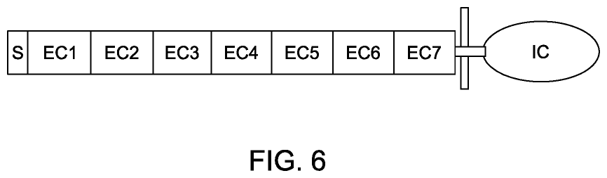

Cadherins are a large group of transmembrane proteins on the cell surface that mediate cell-cell recognition and adhesion (Brasch et al., Trends in Cell Biology, 22:299-310 (2012)). They have a common structural domain called the extracellular cadherin (EC) domain that consists of approximately 110 amino acids; most EC domains have conserved calcium binding sites. The name cadherins thus comes from calcium dependent-adhesive function of these proteins. The cadherins are further classified into subfamilies based on the number and arrangement of EC domains. Thus, the cadherins are subdivided into the classical (type 1) cadherins and closely related (type II) cadherins, desmosomal cadherins and protocadherins (Morishita et al., Current Opinion in Cell Biology, 19:584-592 (2007)). Protocadherins have 6 to 7 EC repeats that have low sequence EC similarities and a divergent cytoplasmic domain compared to classical cadherins. PCDH7 is a 116 kDa protocadherin with 7 EC repeats (FIG. 6). The exact function of protocadherins is unknown but they likely play a role in cell signaling (Halbleib et al., Genes Devel., 20:3199-3214 (2006)).

Although PCDH7 has not be studied in the glomeruli, two studies identified protocadherin 12 in mesangial cells and a related protocadherin called FAT1 in podocytes that may play a role in actin polymerization. Interestingly, protocadherins are mostly expressed in the nervous system (Sano et al., EMBO J, 12:2249-2256 (1993); and Frank et al., Current Opinion in Cell Biology, 14:557-562 (2002)). The recently described antigens, NELL-1 and Sema3B, both are also primarily expressed in the nervous system, and it seems that overexpression of PCDH7 has not been reported in kidney diseases. PCDH7-associated MN appears to be a unique kidney disease associated with overexpression of PCDH7.

As described herein, a novel protein, PCDH7, was identified in a subset of adult patients with PLA2R-negative MN. PCDH7-associated MN appears to be a distinct type of MN.

### Example 2

### Identifying PCDH7-Positive Membranous Nephropathy

A blood sample (e.g., serum) is obtained from a human having membranous nephropathy. The obtained sample is examined for the presence of autoantibodies specific for a PCDH7 polypeptide.

If autoantibodies specific for a PCDH7 polypeptide are detected in the sample, as compared to a control level, then the human can be classified as having a PCDH7-positive membranous nephropathy.

### Example 3

### Treating PCDH7-Positive Membranous Nephropathy

A human identified as having autoantibodies specific for a PCDH7 polypeptide is administered one or more immunosuppressive agents (e.g., corticosteroids, cyclosporine, or a B-cell reduction or depletion agent such as Rituximab).

The administered immunosuppressive agent(s) can reduce inflammation and/or B-cell autoantibody production.

The administered immunosuppressive agent(s) can reduce the level of autoantibodies specific for a PCDH7 polypeptide present within the human.

### Example 4

### Identifying PCDH7-Positive Membranous Nephropathy

A kidney tissue sample is obtained from a human having membranous nephropathy. The obtained sample is examined for an elevated level of a PCDH7 polypeptide.

If an elevated level of a PCDH7polypeptide is detected in the sample, as compared to a control level, then the human can be classified as having a PCDH7-positive membranous nephropathy.

### Example 5

### Treating PCDH7-Positive Membranous Nephropathy

A human identified as having an elevated level of a PCDH7 polypeptide is administered one or more immunosuppressive agents (e.g., corticosteroids, cyclosporine, or a B-cell reduction or depletion agent such as Rituximab).

The administered immunosuppressive agent(s) can reduce inflammation and/or B-cell autoantibody production.

The administered immunosuppressive agent(s) can reduce a level of autoantibodies specific for a PCDH7 polypeptide present within the human.

### Example 6

### Exemplary Embodiments

Embodiment 1. A method for identifying a mammal as having an active membranous nephropathy comprising an elevated level of a polypeptide within kidney tissue of said mammal, wherein said polypeptide is a PCDH7 polypeptide, wherein said method comprises:
(a) determining the presence or absence of autoantibodies within said mammal, wherein said autoantibodies are specific for said polypeptide,
(b) classifying said mammal as having said membranous nephropathy if said autoantibodies are present within said mammal, and
(c) classifying said mammal as not having said membranous nephropathy if said autoantibodies are absent within said mammal.

Embodiment 2. The method of embodiment 1, wherein said mammal is a human.

Embodiment 3. The method of any one of embodiments 1-2, wherein said membranous nephropathy lacks an elevated level of a neural epidermal growth factor (EGF)-like 1 (NELL-1) polypeptide, lacks an elevated level of an EXT1 polypeptide within said kidney tissue, lacks an elevated level of an EXT2 polypeptide within said kidney tissue, and/or lacks an elevated level of a Semaphorin 3B polypeptide within said kidney tissue.

Embodiment 4. The method of any one of embodiments 1-3, wherein said membranous nephropathy lacks an elevated level of a PLA2R polypeptide within said kidney tissue.

Embodiment 5. The method of any one of embodiments 1-4, wherein said membranous nephropathy lacks an elevated level of a THSD7A polypeptide within said kidney tissue.

Embodiment 6. The method of any one of embodiments 1-5, wherein said method comprises detecting the presence of said autoantibodies and classifying said mammal as having said membranous nephropathy.

Embodiment 7. The method of any one of embodiments 1-5, wherein said method comprises detecting the absence of said autoantibodies and classifying said mammal as not having said membranous nephropathy.

Embodiment 8. A method for identifying a mammal as having kidney tissue comprising an elevated level of a polypeptide, wherein said polypeptide is a PCDH7 polypeptide, wherein said method comprises:
(a) determining the presence or absence of said kidney tissue within a sample obtained from said mammal,
(b) classifying said mammal as having said kidney tissue if said presence is determined, and
(c) classifying said mammal as not having said kidney tissue if said absence is determined.

Embodiment 9. The method of embodiment 8, wherein said mammal is a human.

Embodiment 10. The method of any one of embodiments 8-9, wherein said kidney tissue lacks an elevated level of a neural epidermal growth factor (EGF)-like 1 (NELL-1) polypeptide, lacks an elevated level of an EXT1 polypeptide, lacks an elevated level of an EXT2 polypeptide, and/or lacks an elevated level of a Semaphorin 3B polypeptide within said kidney tissue.

Embodiment 11. The method of any one of embodiments 8-10, wherein said kidney tissue lacks an elevated level of a PLA2R polypeptide.

Embodiment 12. The method of any one of embodiments 8-11, wherein said kidney tissue lacks an elevated level of a THSD7A polypeptide.

Embodiment 13. The method of any one of embodiments 8-12, wherein said method comprises detecting said presence and classifying said mammal as having said kidney tissue.

Embodiment 14. The method of any one of embodiments 8-12, wherein said method comprises detecting said absence and classifying said mammal as not having said kidney tissue.

Embodiment 15. A method for identifying a mammal having membranous nephropathy as having autoantibodies specific for a polypeptide, wherein said polypeptide is a PCDH7 polypeptide, wherein said method comprises:
(a) determining the presence or absence of said autoantibodies within said mammal,
(b) classifying said mammal as having said autoantibodies if said autoantibodies are present within said mammal, and
(c) classifying said mammal as not having said autoantibodies if said autoantibodies are absent within said mammal.

Embodiment 16. The method of embodiment 15, wherein said mammal is a human.

Embodiment 17. The method of any one of embodiments 15-16, wherein kidney tissue of said mammal lacks an elevated level of a neural epidermal growth factor (EGF)-like 1 (NELL-1) polypeptide, lacks an elevated level of an EXT1 polypeptide, lacks an elevated level of an EXT2 polypeptide, and/or lacks an elevated level of a Semaphorin 3B polypeptide within said kidney tissue.

Embodiment 18. The method of any one of embodiments 15-17, wherein kidney tissue of said mammal lacks an elevated level of a PLA2R polypeptide.

Embodiment 19. The method of any one of embodiments 15-18, wherein kidney tissue of said mammal lacks an elevated level of a THSD7A polypeptide.

Embodiment 20. The method of any one of embodiments 15-19, wherein said method comprises detecting said presence and classifying said mammal as having said autoantibodies.

Embodiment 21. The method of any one of embodiments 15-19, wherein said method comprises detecting said absence and classifying said mammal as not having said autoantibodies.

Embodiment 22. A method for treating a mammal having membranous nephropathy, wherein said method comprises:
(a) identifying a mammal as having (i) autoantibodies specific for a polypeptide or (ii) kidney tissue comprising an elevated level of said polypeptide, wherein said polypeptide is a PCDH7 polypeptide, and
(b) administering an immunosuppressant to said mammal.

Embodiment 23. The method of embodiment 22, wherein said mammal is a human.

Embodiment 24. The method of any one of embodiments 22-23, wherein said mammal is identified as having said autoantibodies.

Embodiment 25. The method of any one of embodiments 22-24, wherein said mammal is identified as having said kidney tissue.

Embodiment 26. The method of any one of embodiments 22-25, wherein said immunosuppressant is a B-cell inhibitor.

Embodiment 27. The method of embodiment 26, wherein said B-cell inhibitor is rituximab.

Embodiment 28. The method of any one of embodiments 22-25, wherein said immunosuppressant is a calcineurin inhibitor.

Embodiment 29. The method of embodiment 28, wherein said calcineurin inhibitor is cyclosporine or tacrolimus.

Embodiment 30. The method of any one of embodiments 22-25, wherein said immunosuppressant is an mTOR inhibitor.

Embodiment 31. The method of embodiment 30, wherein said mTOR inhibitor is sirolimus or everolimus.

Embodiment 32. The method of any one of embodiments 22-25, wherein said immunosuppressant is a DNA damage inducer.

Embodiment 33.

The method of embodiment 32, wherein said DNA damage inducer is chlorambucil.

Embodiment 34. The method of any one of embodiments 22-33, wherein the level of autoantibodies present within said mammal is reduced by at least 5 percent following said administering step.

Embodiment 35. The method of any one of embodiments 22-33, wherein the level of autoantibodies present within said mammal is reduced by at least 25 percent following said administering step.

Embodiment 36. The method of any one of embodiments 22-33, wherein the level of autoantibodies present within said mammal is reduced by at least 50 percent following said administering step.

Embodiment 37. A method for treating a mammal having membranous nephropathy, wherein said method comprises administering an immunosuppressant to a mammal identified as having (i) autoantibodies specific for a polypeptide or (ii) kidney tissue comprising an elevated level of said polypeptide, wherein said polypeptide is a PCDH7 polypeptide.

Embodiment 38. The method of embodiment 37, wherein said mammal is a human.

Embodiment 39. The method of any one of embodiments 37-38, wherein said mammal was identified as having said autoantibodies.

Embodiment 40. The method of any one of embodiments 37-38, wherein said mammal was identified as having said kidney tissue.

Embodiment 41. The method of any one of embodiments 37-40, wherein said immunosuppressant is a B-cell inhibitor.

Embodiment 42. The method of embodiment 41, wherein said B-cell inhibitor is rituximab.

Embodiment 43. The method of any one of embodiments 37-40, wherein said immunosuppressant is a calcineurin inhibitor.

Embodiment 44. The method of embodiment 43, wherein said calcineurin inhibitor is cyclosporine or tacrolimus.

Embodiment 45. The method of any one of embodiments 37-40, wherein said immunosuppressant is an mTOR inhibitor.

Embodiment 46. The method of embodiment 45, wherein said mTOR inhibitor is sirolimus or everolimus.

Embodiment 47. The method of any one of embodiments 37-40, wherein said immunosuppressant is a DNA damage inducer.

Embodiment 48. The method of embodiment 47, wherein said DNA damage inducer is chlorambucil.

Embodiment 49. The method of any one of embodiments 37-48, wherein the level of autoantibodies present within said mammal is reduced by at least 5 percent following said administering step.

Embodiment 50. The method of any one of embodiments 37-48, wherein the level of autoantibodies present within said mammal is reduced by at least 25 percent following said administering step.

Embodiment 51. The method of any one of embodiments 37-48, wherein the level of autoantibodies present within said mammal is reduced by at least 50 percent following said administering step.

Embodiment 52. A method for treating a mammal having membranous nephropathy and kidney tissue comprising an elevated level of a polypeptide, wherein said polypeptide is a PCDH7 polypeptide, wherein said method comprises administering an immunosuppressant to said mammal.

Embodiment 53. The method of embodiment 52, wherein said mammal is a human.

Embodiment 54. The method of any one of embodiments 52-53, wherein said mammal comprises autoantibodies specific for said polypeptide.

Embodiment 55. The method of any one of embodiments 52-53, wherein said mammal was identified as having said kidney tissue.

Embodiment 56. The method of any one of embodiments 52-55, wherein said kidney tissue lacks an elevated level of a neural epidermal growth factor (EGF)-like 1 (NELL-1) polypeptide.

Embodiment 57. The method of any one of embodiments 52-56, wherein said kidney tissue lacks an elevated level of an EXT1 polypeptide, wherein said kidney tissue lacks an elevated level of an EXT2 polypeptide, and/or wherein said kidney tissue lacks an elevated level of a Semaphorin 3B polypeptide.

Embodiment 58. The method of any one of embodiments 52-57, wherein said kidney tissue lacks an elevated level of a PLA2R polypeptide.

Embodiment 59. The method of any one of embodiments 52-58, wherein said kidney tissue lacks an elevated level of a THSD7A polypeptide.

Embodiment 60. The method of any one of embodiments 52-59, wherein said immunosuppressant is a B-cell inhibitor.

Embodiment 61. The method of embodiment 60, wherein said B-cell inhibitor is rituximab.

Embodiment 62. The method of any one of embodiments 52-59, wherein said immunosuppressant is a calcineurin inhibitor.

Embodiment 63. The method of embodiment 62, wherein said calcineurin inhibitor is cyclosporine or tacrolimus.

Embodiment 64. The method of any one of embodiments 52-59, wherein said immunosuppressant is an mTOR inhibitor.

Embodiment 65. The method of embodiment 64, wherein said mTOR inhibitor is sirolimus or everolimus.

Embodiment 66. The method of any one of embodiments 52-59, wherein said immunosuppressant is a DNA damage inducer.

Embodiment 67. The method of embodiment 66, wherein said DNA damage inducer is chlorambucil.

Embodiment 68. The method of any one of embodiments 52-67, wherein the level of autoantibodies present within said mammal is reduced by at least 5 percent following said administering step.

Embodiment 69. The method of any one of embodiments 52-67, wherein the level of autoantibodies present within said mammal is reduced by at least 25 percent following said administering step.

Embodiment 70. The method of any one of embodiments 52-67, wherein the level of autoantibodies present within said mammal is reduced by at least 50 percent following said administering step.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-14. (canceled)

15. A method for treating a mammal having membranous nephropathy, wherein said method comprises:
(a) identifying a mammal as having (i) autoantibodies specific for a polypeptide or (ii) kidney tissue comprising an elevated level of said polypeptide, wherein said polypeptide is a PCDH7 polypeptide, and
(b) administering an immunosuppressant to said mammal.

16. The method of claim 15, wherein said mammal is a human.

17. The method of claim 15, wherein said mammal is identified as having said autoantibodies.

18. The method of claim 15, wherein said mammal is identified as having said kidney tissue.

19. The method of claim 15, wherein said immunosuppressant is a B-cell inhibitor.

20. (canceled)

21. The method of claim 15, wherein said immunosuppressant is a calcineurin inhibitor.

22. (canceled)

23. The method of claim 15, wherein said immunosuppressant is an mTOR inhibitor.

24. (canceled)

25. The method of claim 15, wherein said immunosuppressant is a DNA damage inducer.

26. The method of claim 15, wherein said immunosuppressant is selected from the group consisting of rituximab, cyclosporine, tacrolimus, sirolimus, everolimus, and chlorambucil.

27. The method of claim 15, wherein the level of autoantibodies present within said mammal is reduced by at least 5 percent following said administering step.

28. A method for treating a mammal having membranous nephropathy, wherein said method comprises administering an immunosuppressant to a mammal identified as having (i) autoantibodies specific for a polypeptide or (ii) kidney tissue comprising an elevated level of said polypeptide, wherein said polypeptide is a PCDH7 polypeptide.

29. The method of claim 28, wherein said mammal is a human.

30. The method of claim 28, wherein said mammal was identified as having said autoantibodies.

31. The method of claim 28, wherein said mammal was identified as having said kidney tissue.

32. The method of claim 28, wherein said immunosuppressant is a B-cell inhibitor.

33. (canceled)

34. The method of claim 28, wherein said immunosuppressant is a calcineurin inhibitor.

35. (canceled)

36. The method of claim 28, wherein said immunosuppressant is an mTOR inhibitor.

37. (canceled)

38. The method of claim 28, wherein said immunosuppressant is a DNA damage inducer.

39. The method of claim 28, wherein said immunosuppressant is selected from the group consisting of rituximab, cyclosporine, tacrolimus, sirolimus, everolimus, and chlorambucil.

40. The method of claim 28, wherein the level of autoantibodies present within said mammal is reduced by at least 5 percent following said administering step.
